(19) United States
(12) Patent Application Publication
(10) Pub. No.: US 2020/0132677 A1
(43) Pub. Date: Apr. 30, 2020

(54) WHOLE BLOOD VOLUME ANALYZER

(71) Applicant: DAXOR CORP., New York, NY (US)

(72) Inventors: Jonathan Feldschuh, Jackson Heights, NY (US); Ronald Cacheiro, Oak Ridge, TN (US)

(73) Assignee: DAXOR CORP., New York, NY (US)

(21) Appl. No.: 16/667,981

(22) Filed: Oct. 30, 2019

Related U.S. Application Data
(60) Provisional application No. 62/753,308, filed on Oct. 31, 2018.
(60) Provisional application No. 62/753,235, filed on Oct. 31, 2018.

(51) Int. Cl. G01N 33/50; G01N 33/80; G01N 33/58; G01N 35/00

(52) U.S. Cl. CPC G01N 33/5094; G01N 33/80; G01N 33/582; G01N 2035/0091; G01N 35/00732; G01N 2035/00752; G01N 33/583

(57) ABSTRACT

Systems and methods are provided for measuring blood volume of a living being.

### CROSS-REFERENCE TO RELATED APPLICATIONS

This application claims the benefit of U.S. Provisional Patent Application No. 62/753,308, filed on Oct. 31, 2018, and the benefit of U.S. Provisional Patent Application No. 62/753,235, filed on Oct. 31, 2018, the contents of which are herein incorporated by reference in their entirety into the present application.

### FIELD OF THE INVENTION (TECHNICAL FIELD)

The present invention relates to systems and methods for measuring blood volume of a living being.

### Background

A blood volume analyzer (BVA) is an instrument or system capable of measuring and reporting the volume of blood of a living being. The Daxor BVA-100 Blood Volume Analyzer, based on U.S. Pat. No. 5,024,231 is a commercially-available, FDA-cleared device. It operates on the indicator-dilution principle. I-131-labelled Human Serum Albumin (HSA) is injected into a patient's blood stream, and various samples of blood are taken at timed intervals after mixing has occurred. These samples must be centrifuged, and precise aliquots of plasma must be taken for counting in the instrument's radiation detector. This adds complexity and operational difficulty, adds time to the procedure, introduces multiple opportunities for error, and prohibits operation of the test at the patient bedside. These factors limit widespread practical clinical use of the blood volume measurement, which has been proven in numerous published clinical studies to have significant health benefits in guiding treatment. Removing some or all of the above factors would solve a major problem.

The reason that samples are centrifuged is two-fold. Firstly, the radiation is contained in the plasma (which makes up approximately 60% of the whole blood), so counting just the plasma is more efficient. The main reason, however, is that quantitative pipetting of whole blood is not recommended, because the cells in the whole blood can clump, adhere to the pipette surfaces or otherwise interfere with the exact function of the pipette, hemolyze, not be distributed evenly in a sample, etc.

The present invention greatly simplifies the performance of a blood volume.

### BRIEF SUMMARY OF EMBODIMENTS OF THE PRESENT INVENTION

The present invention greatly simplifies the performance of a blood volume measurement by employing a flow-through quantitative blood collection vial to allow the use of whole blood directly in the blood volume analyzer. The present invention also has the advantage of using less blood in the performance of a test, as the flow-through design allows the options of returning all blood to the patient, as no blood actually leaves a sterile connection to the patient in the course of measurement. For a typical test performed with the BVA-100, 42 ml of patient blood might be required (6 patient samples, plus a background sample, each 6 ml). The current invention used in closed-loop mode would only consume the blood contained in the external loop (perhaps 6 ml), and even this blood could be flushed with saline and returned to the patient at the end of the procedure if desired.

In one preferred embodiment, the analyzer is a handheld device with an integrated touchscreen, counting well, and barcode reader, which performs measurements of the concentration of a tracer present in samples of whole blood stored in a flow-through quantitative blood collection vial, so as to determine blood volume and associated values.

In another preferred embodiment, a single flow-through vial is used to measure patient background as well as post-injection values, with blood being drawn through the vial using vacuum pressure (via a syringe, pump, or other means).

In another preferred embodiment, the system includes an in-line Hct monitor to ensure that blood being measured is not diluted by saline that is present to keep an IV line open.

In another preferred embodiment, the system includes a kit containing flow-through quantitative blood collection vials to facilitate the performance of a blood volume measurement, said kit including injectate in a vial and a matched standard that can be counted in the analyzer.

### BRIEF DESCRIPTION OF THE SEVERAL VIEWS OF THE DRAWINGS

FIG. 1A-1D shows several views (1A front, 1B side, 1C above, 1D oblique) of one embodiment of the invention, where a handheld analyzer (100) is seen attached to its charging base (101), with a quantitative vial inserted into the counting well of the detector (103), and touchscreen displaying results (102).

FIG. 2A-2E shows several views (2A front, 2B side, 2C oblique, 2D back, 2E above) of the outer casing of the embodiment shown in FIG. 1A-1D, illustrating the counting well opening (200), power button (201), mute button (202) (for silencing alarms, e.g. when a sample should be drawn), barcode scanner button (203), barcode scanner (204), opening for insertion of screen (205), right-side grip (206), and counting well inside left-side grip (207).

FIG. 3A-3B shows two views (3A oblique, 3B side) of the charging base of the embodiment shown in FIG. 1, illustrating the base (300), charging/data connector (301), external connectors (302) for power, data, external printers, etc., and printer paper slot (303) (for printing reports of results).

FIG. 4A-4B shows two views of a preferred embodiment of the invention, in which the barcode scanner is used to read identification data encoded on a quantitative vial.

FIG. 5A-5B show two views of a kit incorporated into a preferred embodiment, in which labelled “standard” (leftmost vial) and “injectate” vials are included in a suitable package such as a foam tray, and encoded with identifying information (such as a barcode, as visible on the underside of the vials in FIG. 5B).

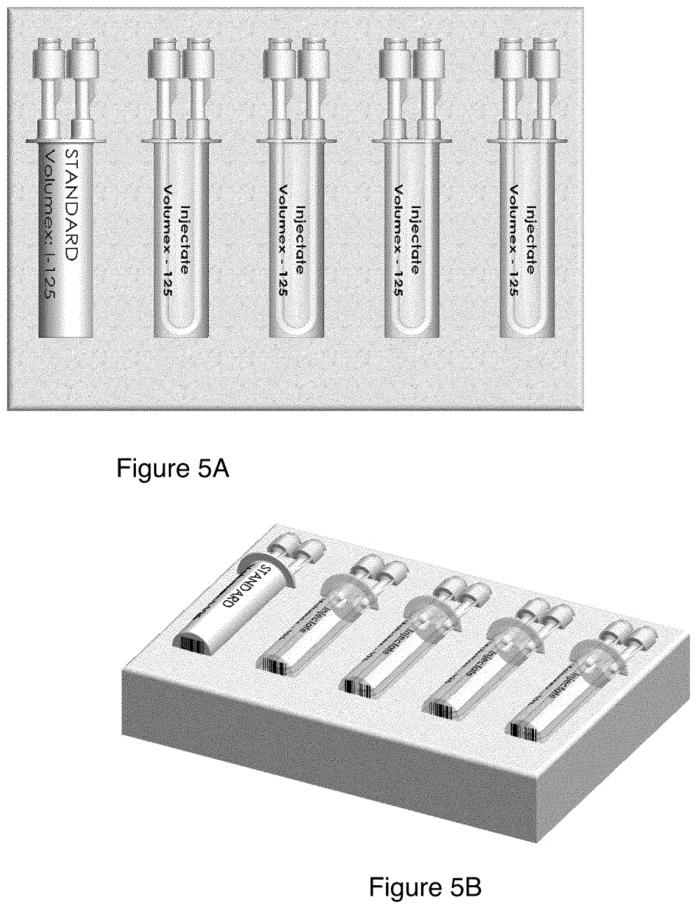

FIG. 6 shows a schematic of one embodiment of the invention. In this embodiment, a patient is connected to a quantitative vial which has been placed in a detector, which is a component of the whole blood volume analyzer system. An in-line Hct monitor ensures that blood coming from the patient is not diluted by saline (which is always present on an IV line to maintain it). A syringe is attached via a stopcock to the egress of the quantitative vial, of sufficient size to hold all the blood that may be required. Once counting has been done, the syringe stopcock is adjusted so that blood drawn may be returned to the patient.

### DETAILED DESCRIPTION OF THE INVENTION

A system is provided for automatically analyzing blood of a living subject, comprising a concentration counter configured to analyze one or more samples, a user interface operatively connected to the concentration counter and configured for entry and display of information, one or more processors operatively coupled to a memory and configured to execute programmed instructions stored in the memory to carry out a method comprising the steps of:
a. measuring a flow-through quantitative blood collection vial containing whole blood from the subject in the concentration counter to determine a background count;
b. injecting the subject with a precise, known volume of tracer;
c. at one or more timed intervals after the injection, drawing blood from the subject into a quantitative flow-through vial, and counting said blood in the detector;
d. calculating, by the one or more processors, a blood volume (BV), plasma volume (PV), and red cell volume (RCV) for the patient;
e. calculating, by the one or more processors, an ideal blood volume (iBV), ideal plasma volume (iPV), and red cell volume (iRCV) for the patient based on patient descriptive data such as height, weight, and gender; and
f. displaying, by the one or more processors, at the user interface, the results.

The calculations in steps (d) and (e) can be performed as follows, for example.

Blood volume can be measured by comparing the counts in a patient sample with the known activity injected into the patient. The known activity can be measured directly, or can be calculated from a ratio of counts in a patient sample to counts observed in a reference standard (the known activity diluted into a known volume).

The overall whole body Hct (oHct) is related to the peripheral Hct by the following relationship:

oHct=pHct*paf (1)

where

paf=0.9009 (2).

This is due to the fact that blood cells are more concentrated in the peripheral circulation (from which blood samples are drawn) than the average value for the whole body; the constant paf is derived as the product 0.99*0.91, as described in U.S. Pat. No. 5,024,231, or a similar constant value. Red Cell Volume and Plasma volume are related to Blood Volume as follows:

BV=PV+RCV (3)

RCV=BV*oHct=BV*pHct*paf (4)

PV=BV*(1−oHct)=BV*(1−pHct*paf) (5).

When the tracer is known to leak out of the bloodstream (as e.g. labelled albumin transudates at a rate of approximately 0.25%/min), a time-zero BV can be calculated using a log-linear regression of BV values obtained at various points.

The Ideal Hct (iHct) is defined to be:

$$iHct ≡ \{ \begin{matrix}0.45 for Males \\ 0.40 for Females\end{matrix} \\ iHct ≡ \{ \begin{matrix}0.45 for Males \\ 0.40 for Females\end{matrix} . \qquad ( 6 )$$

The Ideal Red Cell Volume (iRCV) and Ideal Plasma Volume (iPV) are calculated from the iBV. Note that the iHct is a peripheral Hct value, so the peripheral adjustment factor is required:

iBV=iPV+iRCV (7)

iRCV=iBV*iHct*paf (8)

iPV=iBV−iRCV=iBV*(1−iHct*paf) (9).

The tracer could be a radioactive isotope (such as I-131 or I-125 labelled Human Serum Albumin) or a fluorescent dye (such Indocyanine Green).

In one embodiment, a Hct measuring device is included in the system, to facilitate the calculation above for RCV and PV that include Hct.

A flow-through quantitative blood collection vial is a device for storing, transporting, measuring, and collecting blood, having the properties of a precisely determined volume, the ability to be emptied or filled while connected to a continuous source (such as a supply of a drug, or the bloodstream of a patient), and a geometry suitable for the entire device to be placed in the counting chamber of a detector (such as the counting well of a gamma counter). In one embodiment of the vial, a quantitative flow-through vial is fashioned in a precise, repeatable manner via molding, out a plastic material with suitable handling properties, such that there is a continuous fluid pathway embedded in a retaining structure, with standardized input and output ports, said retaining structure being shaped to fit into a detector (for example, having a test-tube shape matching the dimensions of a counting well in a radiation scintillation detector), with the volume of said vial being precisely known.

The flow-through properties of the vial allow for another embodiment of the present invention, where a single flow-through quantitative blood collection vial is utilized for steps (a) and (c), where said vial is continuously connected to the patient via an IV line, and where sufficient blood is drawn through the vial using vacuum pressure (via a syringe, pump, or other means) to ensure that any saline present in the line is cleared out of the vial and thus does not affect counting accuracy. This allows for a measurement to be made directly at the bedside of a patient.

One issue that arises when blood samples are drawn from an IV line is that saline must always be present in an open IV line to keep it open. When blood is drawn, it is customary to draw sufficient waste blood to ensure that all saline has been cleared and does not affect the sample. In one embodiment of the invention, Hct is monitored by a Hct measuring device. This device could measure continuously (as a Crit-Line monitor functions) or as desired (as a Ultra-Crit device functions). In a preferred embodiment, the system verifies that the Hct measured by the Hct monitor matches a baseline value for the patient to ensure that the sample being counted in the analyzer will result in accurate counts (as opposed to inaccurate, lower counts due to dilution caused by the presence of saline in the vial).

The connection from the patient to the vial can easily be accomplished using all sterile components, as the vials can be sterilized prior to use. In this case, it is possible that all blood sampled from the patient is returned to the patient after being counted by the analyzer. This may be desirable in certain situations, where even the small amounts of blood used in a typical blood volume measurement using this system (perhaps 20-30 ml) might be significant for a given patient. In one embodiment, blood is returned to the patient after passing through sterile vials.

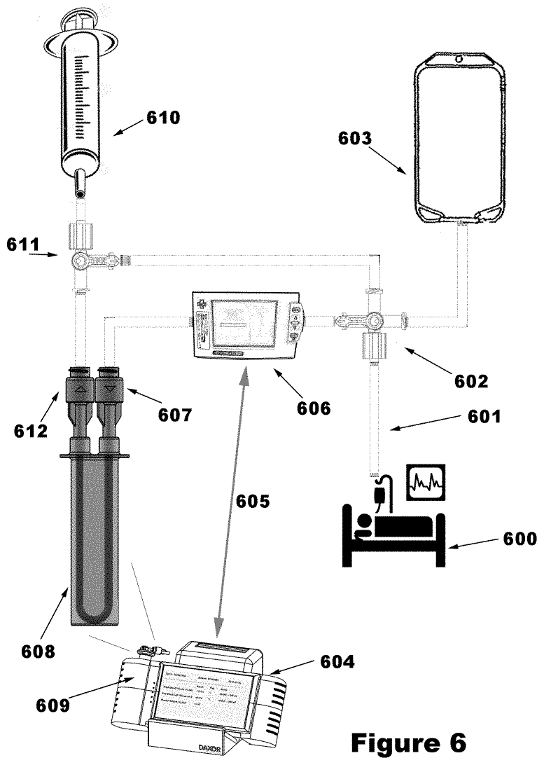

FIG. 6 illustrates a preferred embodiment. A patient (600) has an IV line (601) attached through a 4-way stopcock (602), and receives saline (603) when the stopcock (602) is positioned accordingly. An analyzer (604) is operatively connected (605) with a Hct monitor (606) which is also connected to the 4-way stopcock (602). The Hct Monitor is connected to the ingress (607) of a quantitative flow-through vial (608) which is inserted into the counting well of the analyzer (609). When a sample is to be collected for measurement, the syringe (610), connected to the egress (612) of the vial (608) via a 3-way stopcock (611), creates vacuum pressure as the plunger is retracted, causing the vial to be filled with blood from the patient, with any excess blood entering the syringe. As the blood passes through the Hct monitor (606), the analyzer confirms that the Hct reading of the blood matches a baseline value for the patient, indicating that measurement can commence once a sufficient extra volume of blood corresponding to the volume of tubing between the Hct monitor and the vial has been drawn. Multiple samples can be measured in this fashion. After all desired measurements are complete, the blood drawn into the syringe in the course of the procedure can be returned to the patient by setting the stopcocks (611, 602) accordingly and depressing the syringe plunger.

The use of vacuum pressure to draw blood into the vial enables another embodiment, where the drawing of blood in steps (a) and/or steps (c) is accomplished by means of an automated pump that is controlled by the analyzer system. This has the important advantage of enabling the performance of a blood volume measurement with less operator intervention. Once a patient has been injected with the tracer, the system can draw samples at desired time intervals (e.g. 12, 18, 24, 30, and 36 minutes) without requiring further action from the operator.

In one embodiment, the calculation results from step (e) are reported at the user interface immediately after the first sample is counted in step (c), and updated after each iteration of step (c). This has the benefit of providing preliminary information (which in certain situations might be actionable) while still providing eventual confirmation and additional information based on the calculation of a multi-point versus a single-point estimation of blood volume. E.g. in the case described in the preceding paragraph where five timed samples between 12 and 36 minutes are used, a first preliminary blood volume might be calculated after approximately 16 minutes (after four minutes of counting of the 12 minute sample), with the final calculation reported after 40 minutes.

The flow-through design of the vials allows for injectate to be precisely delivered to a patient after being stored. In one embodiment of the present invention, the injectate is counted directly in the analyzer before injection.

For many tracers, this embodiment will not be feasible, as many detection systems (such as scintillation systems for radiation detection) do not possess sufficient range of linearity to measure an injectate which might be 8000 times as concentrated as the diluted patient sample (as for example, would occur if a 1 ml injectate were injected into a patient with a blood volume of 8000 ml). To handle this use case, a reference standard is generally employed, where an identical injectate is diluted to a known volume (e.g. 1000 ml). In one embodiment, a calibrated standard prepared (by precise dilution into a known volume) from the same manufacturing lot as the injectate in step (b) is contained in a dimensionally identical quantitative vial and is measured in the detector before the injection to determine the quantity of injectate.

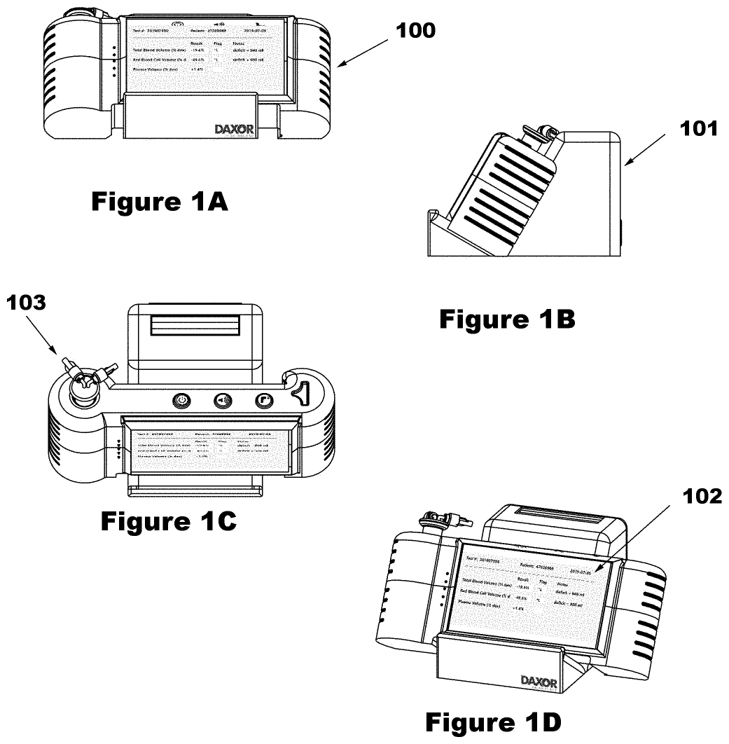

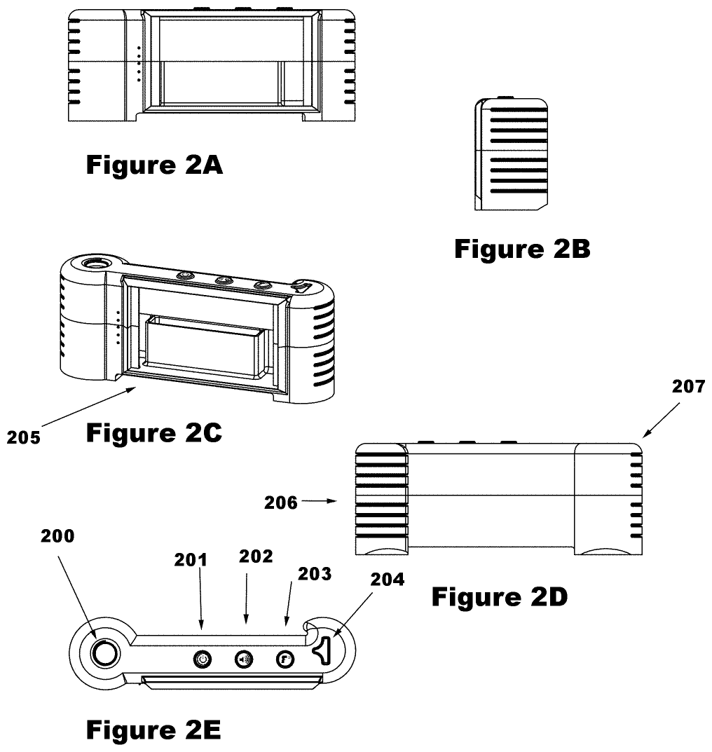

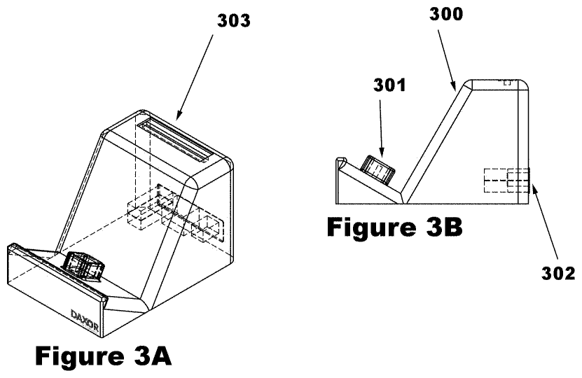

In one preferred embodiment, the analyzer is a handheld device containing a detector module and a touchscreen interface, with an associated charging base. As described above, FIGS. 1A-3B illustrate features of such a device.

In another preferred embodiment, this analyzer is used as part of a method for measuring human blood volume. The method, for example, can comprise the steps of:
a. measuring a flow-through quantitative blood collection vial containing whole blood from the subject in the concentration counter to determine a background count;
b. injecting the subject with a precise, known volume of tracer;
c. at one or more timed intervals after the injection, drawing blood from the subject into a quantitative flow-through vial, and counting said blood in the detector; and
d. calculating a blood volume (BV), plasma volume (PV), and red cell volume (RCV) for the patient.

In another preferred embodiment, the method for measuring human blood volume additionally employs a kit consisting of a number of dimensionally equivalent quantitative vials, where one of the vials contains an injectate used in step (1)(b), and one of the vials contains a calibrated standard prepared (by precise dilution into a known volume) from the same manufacturing lot as the injectate, where the standard vial is measured in the detector before the injection to determine the precise volume of tracer injected in step (1)(b). The identical geometry of the standard vial and the patient collection vial ensures accurate comparison of activity and hence volume.

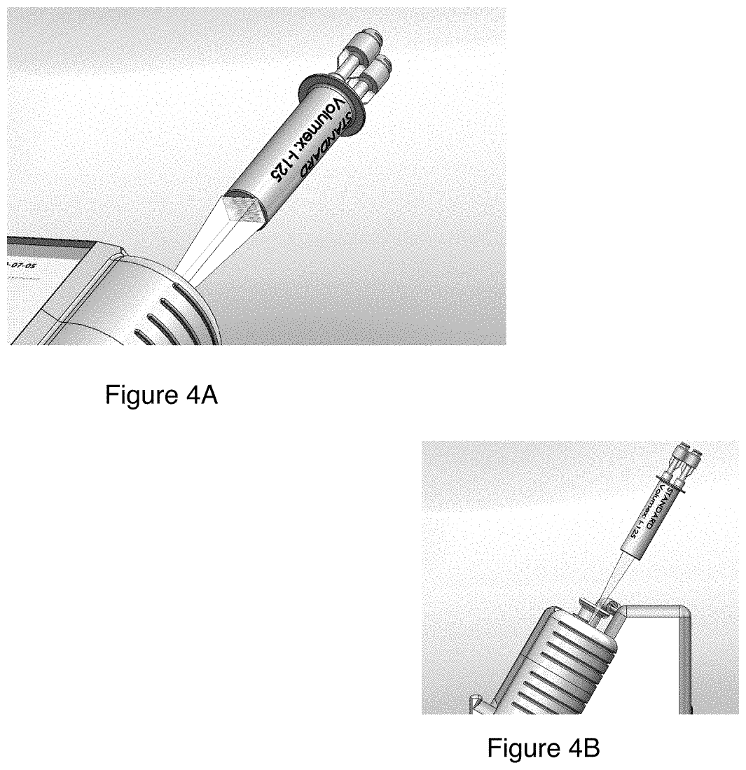

In another preferred embodiment, the barcode reader operatively connected to or integrated with the analyzer is used to facilitate the method, by ensuring that standard and injectate bear the same matching identification, and allowing the standard to be optionally counted (with the results stored in the analyzer, or in a data store operatively connected to the analyzer) prior to any interaction with a subject, shortening the time necessary to achieve a measurement. For example, as seen in FIGS. 4A and 4B, the barcode reader is used to scan information contained on the standard vial. This information includes a unique identifier that matches injectate vials from the same lot. Scanning the standard and injectate ensures that the measurement is accurate. In addition, because the radioactive decay constant of the injectate can also be coded into the information, it is possible to measure the activity of the standard in advance of the patient measurement (e.g. at the start of a day as part of quality control), and to store that measurement for use later. If desired, the earlier measurement can be adjusted for the drop in observed counts due to elapsed time, and used in conjunction with a test performed later using matching injectate. This has the benefit of reducing the time needed and number of steps necessary when a blood volume measurement is performed on a patient.

What is claimed is:

1. A system for automatically analyzing blood of a living subject, comprising a concentration counter configured to analyze one or more samples, a user interface operatively connected to the concentration counter and configured for entry and display of information, one or more processors operatively coupled to a memory and configured to execute programmed instructions stored in the memory to carry out a method comprising the steps of:
a. measuring a flow-through quantitative blood collection vial containing whole blood from the subject in the concentration counter to determine a background count;
b. injecting the subject with a precise, known volume of tracer;
c. at one or more timed intervals after the injection, drawing blood from the subject into a quantitative flow-through vial, and counting said blood in the detector;
d. calculating, by the one or more processors, a blood volume (BV), plasma volume (PV), and red cell volume (RCV) for the patient;
e. calculating, by the one or more processors, an ideal blood volume (iBV), ideal plasma volume (iPV), and red cell volume (iRCV) for the patient based on patient descriptive data such as height, weight, and gender; and
f. displaying, by the one or more processors, at the user interface, the results.

2. The system of claim 1, where the tracer is a radioactive isotope, and the counter is a radiation counter.

3. The system of claim 1, where the tracer is a light-emitting (fluorescent) or light-absorbent (dye), and the counter is capable of measuring light emission or absorption.

4. The system of claim 1, where a single flow-through quantitative blood collection vial is utilized for steps (a) and (c), where said vial is continuously connected to the patient via an IV line, and where sufficient blood is drawn through the vial using vacuum pressure (via a syringe, pump, or other means) to ensure that any saline present in the line is cleared out of the vial and thus does not affect counting accuracy.

5. The system of claim 4, where a hematocrit measuring device is a component of the system.

6. The system of claim 5, where the hematocrit measuring device is used to verify that the line is sufficiently clear of saline (by comparing the current measurement of Hct to a previous or baseline value) in steps (a) and/or steps (c).

7. The system of claim 4, where the quantitative vials are sterile, and blood is returned to the patient after it has passed through the quantitative vial after steps (a) and/or steps (c).

8. The system of claim 4, where the drawing of blood in steps (a) and/or steps (c) is accomplished by means of an automated pump that is controlled by the analyzer system.

9. The system of claim 1, where interim results from step (e) are reported at the user interface immediately after the first sample is counted in step (c), and updated after each iteration of step (c).

10. The system of claim 1, where the injectate in step (b) is contained in an identical quantitative vial and is measured in the detector before the injection to determine the quantity of injectate.

11. The system of claim 1, where a calibrated standard is prepared by precise dilution into a known volume of injectate from the same manufacturing lot as the injectate in step (b), is contained in a dimensionally identical quantitative vial, and is measured in the detector to determine the quantity of injectate.

12. The system of claim 1, where the analyzer is a handheld device containing a detector module and a touchscreen interface, with an associated charging base.

13. The system of claim 13, where the analyzer includes an integrated barcode reader capable of reading codes on vials.

14. A method of measuring the blood volume of a subject using the system of claim 1, the method comprising the steps of:
a. measuring a flow-through quantitative blood collection vial containing whole blood from the subject in the concentration counter to determine a background count;
b. injecting the subject with a precise, known volume of tracer;
c. at one or more timed intervals after the injection, drawing blood from the subject into a quantitative flow-through vial, and counting said blood in the detector; and
d. calculating a blood volume (BV), plasma volume (PV), and red cell volume (RCV) for the patient.

15. The method of claim 14, using in addition a kit consisting of a number of dimensionally equivalent quantitative vials, where one of the vials contains an injectate used in step (14)(b), and the injectate vial is measured directly in the analyzer to determine the precise volume of tracer injected in step (14)(b).

16. The method of claim 14, using in addition a kit consisting of a number of dimensionally equivalent quantitative vials, where one of the vials contains an injectate used in step (14)(b), and one of the vials contains a calibrated standard prepared (by precise dilution into a known volume) from the same manufacturing lot as the injectate, where the standard vial is measured in the detector before the injection to determine the precise volume of tracer injected in step (14)(b).

17. The method of claim 14, where a barcode reader operatively connected to or integrated with the analyzer is used to facilitate the method, by ensuring that standard and injectate bear the same matching identification, and allowing the standard to be optionally counted (with the results stored in the analyzer, or in a data store operatively connected to the analyzer) prior to any interaction with a subject, shortening the time necessary to achieve a measurement.
